(19) United States
(12) Patent Application Publication
(10) Pub. No.: US 2024/0000395 A1
(43) Pub. Date: Jan. 4, 2024

(54) HEART RATE ESTIMATION METHOD, DEVICE, AND COMPUTER-READABLE STORAGE MEDIUM

(71) Applicant: BOMDIC INC., New Taipei City (TW)

(72) Inventor: Wei-Chiao Chang, New Taipei City (TW)

(73) Assignee: BOMDIC INC., New Taipei City (TW)

(21) Appl. No.: 17/852,389

(22) Filed: Jun. 29, 2022

(51) Int. Cl. A61B 5/00; A61B 5/0205; A61B 5/11

(52) U.S. Cl. CPC A61B 5/7278; A61B 5/0205; A61B 5/7203; A61B 5/1114; A61B 5/02416

(57) ABSTRACT

Embodiments of the disclosure provide a heart rate estimation method and device and a computer-readable storage medium. The method includes: obtaining a reference photoplethysmography (PPG) spectrum corresponding to a t-th time point; obtaining a previous heart rate and a motion energy parameter corresponding to the t-th time point, and accordingly determining a predicted heart rate corresponding to the t-th time point; determining a reference mask based on the predicted heart rate and the motion energy parameter of the t-th time point; determining a specific PPG spectrum based on the reference mask and the reference PPG spectrum; and estimating a heart rate corresponding to the t-th time point based on the specific PPG spectrum.

### TECHNICAL FIELD

The disclosure relates to a physiological state estimation technique, and more particularly to a heart rate estimation method and device and a computer-readable storage medium.

### DESCRIPTION OF RELATED ART

In modern society, it is quite common to detect the physiological states (for example, blood pressure, heart rate, etc.) of people through wearable devices. However, during the process of detecting the heart rate of the user by the wearable device, there may be cases where the heart rate is not detected for a certain period of time due to unstable signal, noise in signal, the wearable device not properly worn, or the exercise state of the user.

### SUMMARY

In view of this, the disclosure provides a heart rate estimation method and device and a computer-readable storage medium, which can be used to solve the above-mentioned technical issue.

An embodiment of the disclosure provides a heart rate estimation method, which is suitable for a heart rate estimation device and includes: obtaining a reference photoplethysmography spectrum corresponding to a t-th time point, where t is a time index value; obtaining a previous heart rate and a motion energy parameter corresponding to the t-th time point, and accordingly determining a predicted heart rate corresponding to the t-th time point; determining a reference mask based on the predicted heart rate and the motion energy parameter of the t-th time point; determining a specific photoplethysmography spectrum based on the reference mask and the reference photoplethysmography spectrum; and estimating a heart rate corresponding to the t-th time point based on the specific photoplethysmography spectrum.

An embodiment of the disclosure provides a heart rate estimation device, which includes a storage circuit and a processor. The storage circuit stores a code. The processor is coupled to the storage circuit and accesses the code to execute: obtaining a reference photoplethysmography spectrum corresponding to a t-th time point, where t is a time index value; obtaining a previous heart rate and a motion energy parameter corresponding to the t-th time point, and accordingly determining a predicted heart rate corresponding to the t-th time point; determining a reference mask based on the predicted heart rate and the motion energy parameter of the t-th time point; determining a specific photoplethysmography spectrum based on the reference mask and the reference photoplethysmography spectrum; and estimating a heart rate corresponding to the t-th time point based on the specific photoplethysmography spectrum.

An embodiment of the disclosure provides a computer-readable storage medium. The computer-readable storage medium records an executable computer program. The executable computer program is loaded by a heart rate estimation device to execute: obtaining a reference photoplethysmography spectrum corresponding to a t-th time point, where t is a time index value; obtaining a previous heart rate and a motion energy parameter corresponding to the t-th time point, and accordingly determining a predicted heart rate corresponding to the t-th time point; determining a reference mask based on the predicted heart rate and the motion energy parameter of the t-th time point; determining a specific photoplethysmography spectrum based on the reference mask and the reference photoplethysmography spectrum; and estimating a heart rate corresponding to the t-th time point based on the specific photoplethysmography spectrum.

### BRIEF DESCRIPTION OF THE DRAWINGS

FIG. 1 is a schematic diagram of a heart rate estimation device according to an embodiment of the disclosure.

FIG. 2 is a flowchart of a heart rate estimation method according to an embodiment of the disclosure.

FIG. 3 is a schematic diagram of determining a predicted heart rate corresponding to a t-th time point based on a reference value according to an embodiment of the disclosure.

FIG. 4 is a schematic diagram of determining a specific photoplethysmography spectrum according to an embodiment of the disclosure.

### DETAILED DESCRIPTION OF DISCLOSED EMBODIMENTS

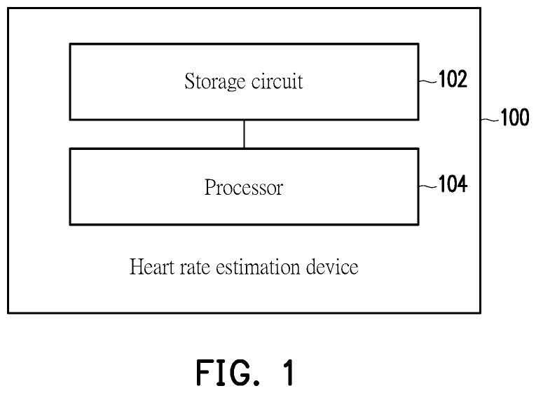

Please refer to FIG. 1, which is a schematic diagram of a heart rate estimation device according to an embodiment of the disclosure. In different embodiments, a heart rate estimation device 100 may be implemented as various smart devices and/or computer devices. In an embodiment, the heart rate estimation device 100 may, for example, obtain various electronic signals (for example, acceleration data, a photoplethysmography (PPG) signal, etc.) measured by a wearable device from the wearable device worn on the body of the user to be analyzed accordingly, so as to estimate the physiological state (for example, the heart rate) of the user. In another embodiment, the heart rate estimation device 100 may be, for example, a combination of a smart device and/or a computer device and a wearable sensor worn on the body of the user, and the device obtains various electronic signals measured by the wearable sensor to be analyzed. In yet another embodiment, the acceleration data may be obtained by a sensor other than the heart rate estimation device 100, such as an acceleration sensor. In addition, the acceleration data may also be the acceleration data obtained by a single sensor, or the acceleration data obtained by multiple sensors to comprehensively evaluate the exercise or activity state of the user.

In some embodiments, the heart rate estimation device 100 may also be implemented as various wearable devices, such as a wristband, a watch, a ring, a necklace, an earphone, and glasses, and may detect the above-mentioned electronic signal through a relevant detection circuit (for example, a PPG signal transceiver, an accelerometer, etc.) when worn on the body of the user. Afterwards, the heart rate estimation device 100 may perform analysis based on the detected electronic signal to estimate the physiological state (for example, the heart rate) of the user. For the convenience of description, it is assumed that the heart rate estimation device 100 is a wearable device worn on the body of the user, but only used as an example and not intended to limit possible implementations of the disclosure.

In FIG. 1, the heart rate estimation device 100 includes a storage circuit 102 and a processor 104. The storage circuit 102 is, for example, any type of fixed or removable random access memory (RAM), read-only memory (ROM), flash memory, hard disk drive, other similar devices, or a combination of the devices, and may be used to record multiple codes or modules.

The processor 104 is coupled to the storage circuit 102 and may be a general-purpose processor, a specific-purpose processor, a conventional processor, a digital signal processor, multiple microprocessors, one or more microprocessors combined with a digital signal processor core, a controller, a microcontroller, an application specific integrated circuit (ASIC), a field programmable gate array (FPGA), any other type of integrated circuit, state machine, processor based on an advanced RISC machine (ARM), and the like.

In the embodiment of the disclosure, the processor 104 may access the module and the code recorded in the storage circuit 102 to implement the heart rate estimation method provided by the disclosure, and the details thereof are described below.

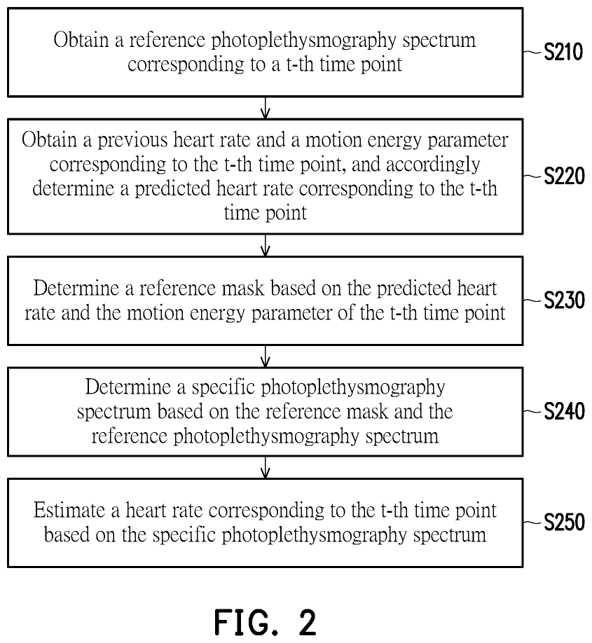

Please refer to FIG. 2, which is a flowchart of a heart rate estimation method according to an embodiment of the disclosure. The method of the embodiment may be executed by the heart rate estimation device 100 of FIG. 1, and the details of each step of FIG. 2 will be described below in conjunction with the elements shown in FIG. 1.

In Step S210, the processor 104 obtains a reference photoplethysmography spectrum corresponding to a t-th time point. In an embodiment, the reference photoplethysmography spectrum is, for example, a photoplethysmography spectrum after de-noising processing.

As previously mentioned, the heart rate estimation device 100 may obtain various electronic signals, such as the photoplethysmography signal and the acceleration data, measured from the user. In an embodiment of the disclosure, the processor 104 may, for example, convert the photoplethysmography signal into a corresponding photoplethysmography spectrum through fast Fourier transform or similar transformation. Afterwards, the processor 104 may, for example, perform the de-noising processing on the photoplethysmography spectrum based on the acceleration data corresponding to the photoplethysmography signal (for example, corresponding to the same time interval).

In an embodiment, for the details of the de-noising processing, reference may be made to “Temko, Andriy. “Accurate heart rate monitoring during physical exercises using PPG.” IEEE Transactions on Biomedical Engineering 64.9 (2017): 2016-2024” (hereinafter referred to as Citation 1), which will not be repeated here.

In Step S220, the processor 104 obtains a previous heart rate (hereinafter referred to as LastHR) and a motion energy parameter (hereinafter referred to as ACₜ) corresponding to the t-th time point, and determines a predicted heart rate (hereinafter referred to as PredictedHR(t)) corresponding to the t-th time point accordingly.

In some embodiments, the acceleration data obtained by the heart rate estimation device 100 is, for example, acceleration values on multiple axes, and the acceleration values may vary in response to correspond to the motion of the wearable device (for example, the heart rate estimation device 100) worn on the user to reflect the exercise or activity state of the user. Therefore, after obtaining the acceleration values of the t-th time point, the processor 104 may determine an original motion energy parameter (hereinafter referred to as ACₜ^{raw}) of the t-th time point accordingly. In an embodiment, the processor 104 may, for example, estimate a corresponding activity count based on the acceleration values corresponding to the t-th time point as the original motion energy parameter of the t-th time point, but not limited thereto. In an embodiment, the processor 104 may, for example, integrate the acceleration values corresponding to the t-th time point to obtain the activity count, but not limited thereto.

Afterwards, the processor 104 may obtain multiple historical motion energy parameters corresponding to a (t-k)-th time point to a (t−1)-th time point, where k is a window length. Afterwards, the processor 104 may determine the motion energy parameter corresponding to the t-th time point based on the original motion energy parameter corresponding to the t-th time point and the historical motion energy parameter.

In an embodiment, the motion energy parameter corresponding to the t-th time point is, for example, a weighted result of the original motion energy parameter and the historical motion energy parameter.

In an embodiment, the motion energy parameter corresponding to the t-th time point may be, for example, represented as “ACₜ=ACₜ^{raw}+Σᵢ₌₁^{k}aᵢACₜ₋ᵢ”, where ACₜ₋ᵢ is the historical motion energy parameter of a (t−i)-th time point, and aᵢ is a coefficient corresponding to ACₜ₋ᵢ (which may be determined by the designer according to the requirements), but not limited thereto.

In an embodiment, the previous heart rate is, for example, a heart rate corresponding to a (t−j)-th time point, where j is a positive integer. In an embodiment, j is, for example, 1, that is, the processor 104 may obtain the heart rate of the (t−1)-th time point as the previous heart rate, but not limited thereto.

In an embodiment, the processor 104 may determine a reference value (hereinafter referred to as A(t)) based on the previous heart rate (that is, LastHR) and the motion energy parameter (that is, ACₜ) corresponding to the t-th time point. In an embodiment, A(t) may be represented as “A(t)=ACₜ*w1+LastHR*w2+c”, where c is a constant, w1 and w2 are coefficients, 0≤w1≤1, and 0≤w2≤1, but not limited thereto.

Thereafter, the processor 104 may determine the predicted heart rate (that is, PredictedHR(t)) corresponding to the t-th time point based on the reference value (that is, A(t)).

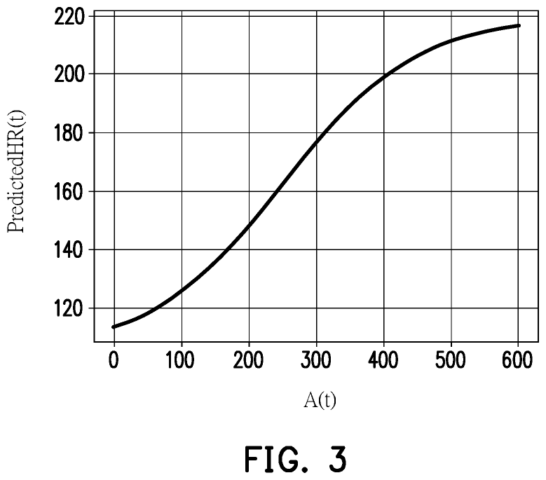

Please refer to FIG. 3, which is a schematic diagram of determining the predicted heart rate corresponding to the t-th time point based on the reference value according to an embodiment of the disclosure.

In FIG. 3, the relationship between PredictedHR(t) and A(t) may be, for example represented as a curve as shown in FIG. 3. Based on this, after obtaining A(t) through the above teaching, the processor 104 may estimate the corresponding value of PredictedHR(t) based on FIG. 3. For example, if A(t) is 400, then PredictedHR(t) is about 200; and if A(t) is 300, then PredictedHR(t) is about 178, but not limited thereto. In an embodiment, A(t) is positively correlated with PredictedHR(t). That is, the greater the A(t), the greater the PredictedHR(t), and vice versa.

In other embodiments, the curve of FIG. 3 may also be recorded as a corresponding look-up table. Based on this, after obtaining A(t), the processor 104 may find the value corresponding to A(t) in the look-up table as PredictedHR(t), but not limited thereto.

In Step S230, the processor 104 determines a reference mask based on the predicted heart rate and the motion energy parameter of the t-th time point.

In an embodiment, the processor 104 may determine a reference standard deviation (hereinafter referred to as SD(t)) based on the motion energy parameter (that is, ACₜ) of the t-th time point, wherein the reference standard deviation is negatively correlated with the motion energy parameter of the t-th time point. That is, the greater the ACₜ, the smaller the reference standard deviation, and vice versa.

After that, the processor 104 may determine a reference normal distribution curve as the reference mask based on the predicted heart rate (that is, PredictedHR(t)) and the reference standard deviation (that is, SD(t)) of the t-th time point, wherein the mean and the standard deviation of the reference normal distribution curve are respectively the predicted heart rate and the reference standard deviation of the t-th time point.

It can be seen from the above that if the estimated ACₜ is greater, the reference standard deviation will be smaller, such that the reference normal distribution curve will appear narrower/higher. On the other hand, if the estimated ACₜ is smaller, the reference standard deviation will be greater, such that the reference normal distribution curve will appear wider/lower.

In Step S240, the processor 104 determines a specific photoplethysmography spectrum based on the reference mask and the reference photoplethysmography spectrum.

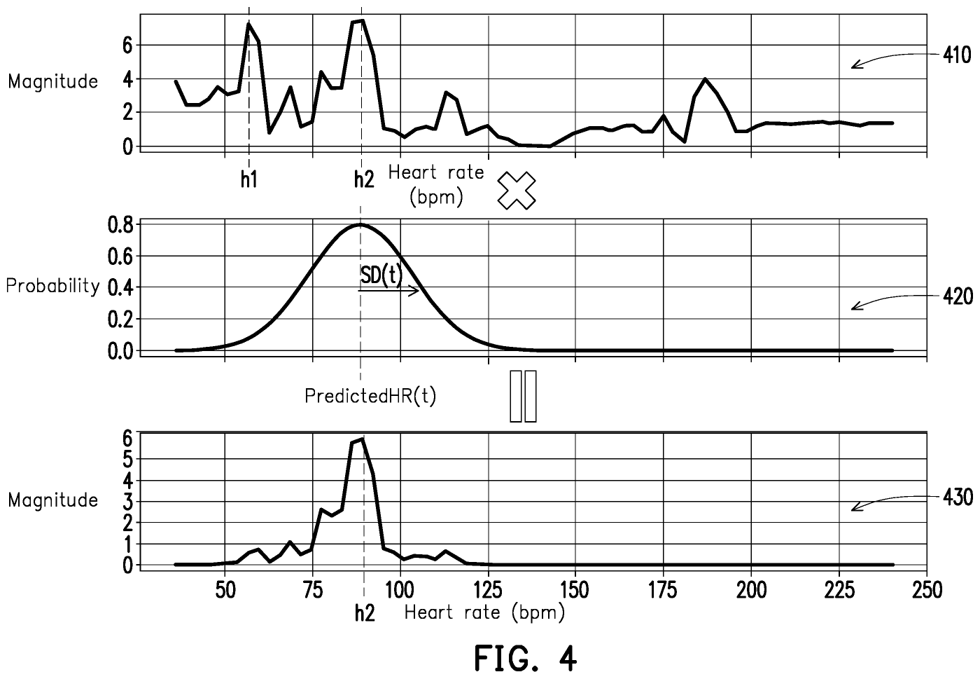

Please refer to FIG. 4, which is a schematic diagram of determining the specific photoplethysmography spectrum according to an embodiment of the disclosure. In FIG. 4, it is assumed that the processor 104 obtains a reference photoplethysmography spectrum 410 (that is, the photoplethysmography spectrum after the de-noising processing) in Step S210, and obtains a reference mask 420 (that is, the reference normal distribution curve with PredictedHR(t) and the reference standard deviation SD(t) as the mean and the standard deviation respectively) in Step S230.

Afterwards, the processor 104 may, for example, multiply the reference mask 420 by the photoplethysmography spectrum 410 to generate a specific photoplethysmography spectrum 430. In FIG. 4, the processor 104 may, for example, multiply the reference mask 420 by the photoplethysmography spectrum 410 by means of point-by-point multiplication to generate the specific photoplethysmography spectrum 430, but not limited thereto.

After that, in Step S250, the processor 104 estimates the heart rate (represented as HR(t)) corresponding to the t-th time point based on the specific photoplethysmography spectrum 430.

In an embodiment, the processor 104 may, for example, estimate the heart rate corresponding to the t-th time point based on the specific photoplethysmography spectrum 430 based on the manner disclosed in Citation 1, and for the relevant details thereof, reference may be made to the content of Citation 1, which will not be repeated here.

In the scenario of FIG. 4, HR(t) obtained by the processor 104 corresponds, for example, to the heart rate with the highest magnitude in the specific photoplethysmography spectrum 430, such as the heart rate corresponding to the magnitude of 6 (about 90 bpm).

Further, if the processor 104 directly executes the heart rate estimation behavior in Citation 1 based on the photoplethysmography spectrum 410, the processor 104 may obtain an erroneous heart rate estimation value due to the presence of multiple heart rates corresponding to greater magnitudes in the photoplethysmography spectrum 410. For example, since heart rates h1 and h2 in the photoplethysmography spectrum 410 both correspond to greater magnitudes, if the processor 104 directly performs heart rate estimation with the photoplethysmography spectrum 410, the less accurate heart rate h1 may be used as HR(t).

However, after performing an operation similar to masking on the photoplethysmography spectrum 410 based on the reference mask 420 to generate the specific photoplethysmography spectrum 430, it can be seen that the magnitude corresponding to the heart rate h1 in the specific photoplethysmography spectrum 430 is significantly reduced. Based on this, when the processor 104 performs the heart rate estimation based on the specific photoplethysmography spectrum 430, the probability of mistakenly using the heart rate h1 as HR(t) can be avoided, thereby improving the accuracy of the heart rate estimation.

In an embodiment, when the processor 104 is used to estimate a heart rate (represented as HR(t+1)) of a (t+1)-th time point, HR(t) may be used as the previous heart rate (that is, LastHR of the (t+1)-th time point), and ACₜ may be used as one of the components to estimate the motion energy parameter corresponding to the (t+1)-th time point.

In an embodiment, the motion energy parameter corresponding to the (t+1)-th time point may be, for example, represented as “ACₜ₊₁=ACₜ^{raw}+Σᵢ₌₁^{k}aⁱACₜ₋ᵢ₊₁”, where ACₜ₊₁^{raw} is, for example, the original motion energy parameter of the (t+1)-th time point. After that, the processor 104 may determine a reference standard deviation (hereinafter referred to as SD(t+1)) corresponding to the (t+1)-th time point based on ACₜ₊₁.

In addition, a reference value (represented as A(t+1)) corresponding to the (t+1)-th time point may be correspondingly represented as “A(t+1)=ACₜ₊₁*w1+LastHR*w2+c”. Afterwards, the processor 104 may, for example, determine a predicted heart rate (represented as PredictedHR(t+1)) corresponding to the (t+1)-th time point based on FIG. 3, and correspondingly determine a reference mask corresponding to the (t+1)-th time point based on PredictedHR(t+1) and SD(t+1).

Based on this, the processor 104 may then generate a specific photoplethysmography spectrum corresponding to the (t+1)-th time point based on the above teaching, thereby estimating a heart rate (represented as HR(t+1)) corresponding to the (t+1)-th time point accordingly. For relevant details, reference may be made to the descriptions in the above embodiments, which will not be repeated here.

In addition, the disclosure further provides a computer-readable storage medium for executing the heart rate estimation method. The computer-readable storage medium is composed of multiple program instructions (for example, a setting program instruction and a deployment program instruction) implemented therein. The program instructions may be loaded to the heart rate estimation device 100 and executed by the heart rate estimation device 100, so as to execute the heart rate estimation method and functions of the heart rate estimation device 100.

In summary, the method provided by the disclosure can determine the reference mask based on the predicted heart rate and the motion energy parameter of the t-th time point, and use the reference mask to suppress the magnitudes of certain heart rates (for example, less accurate heart rates) in the reference photoplethysmography spectrum, thereby generating the specific photoplethysmography spectrum. In this way, the heart rate estimation device according to the embodiments of the disclosure can estimate a relatively accurate heart rate of the user based on the specific photoplethysmography spectrum.

Although the disclosure has been disclosed in the above embodiments, the embodiments are not intended to limit the disclosure. Persons skilled in the art may make some changes and modifications without departing from the spirit and scope of the disclosure. Therefore, the protection scope of the disclosure shall be defined by the appended claims.

What is claimed is:

1. A heart rate estimation method, suitable for a heart rate estimation device, comprising:
obtaining a reference photoplethysmography spectrum corresponding to a t-th time point, where t is a time index value;
obtaining a previous heart rate and a motion energy parameter corresponding to the t-th time point, and accordingly determining a predicted heart rate corresponding to the t-th time point;
determining a reference mask based on the predicted heart rate and the motion energy parameter of the t-th time point;
determining a specific photoplethysmography spectrum based on the reference mask and the reference photoplethysmography spectrum; and
estimating a heart rate corresponding to the t-th time point based on the specific photoplethysmography spectrum.

2. The method according to claim 1, wherein the reference photoplethysmography spectrum is a photoplethysmography spectrum after de-noising processing.

3. The method according to claim 1, wherein the step of obtaining the motion energy parameter corresponding to the t-th time point comprises:
obtaining an original motion energy parameter corresponding to the t-th time point;
obtaining a plurality of historical motion energy parameters corresponding to a (t−k)-th time point to a (t−1)-th time point, where k is a window length;
determining the motion energy parameter corresponding to the t-th time point based on the original motion energy parameter corresponding to the t-th time point and the historical motion energy parameters.

4. The method according to claim 3, wherein the motion energy parameter corresponding to the t-th time point is a weighted result of the original motion energy parameter and the historical motion energy parameters.

5. The method according to claim 1, wherein the previous heart rate is a heart rate corresponding to a (t−j)-th time point, where j is a positive integer.

6. The method according to claim 1, wherein the step of determining the predicted heart rate corresponding to the t-th time point comprises:
determining a reference value based on the previous heart rate and the motion energy parameter corresponding to the t-th time point; and
determining the predicted heart rate corresponding to the t-th time point based on the reference value, wherein the reference value is positively correlated with the predicted heart rate corresponding to the t-th time point.

7. The method according to claim 6, wherein the reference value is represented as:
A(t)=ACₜ*w1+LastHR*w2+c,
where ACₜ is the motion energy parameter corresponding to the t-th time point, LastHR is the previous heart rate, c is a constant, w1 and w2 are coefficients, 0≤w1, and w2≤1.

8. The method according to claim 1, wherein the step of determining the reference mask based on the predicted heart rate and the motion energy parameter of the t-th time point comprises:
determining a reference standard deviation based on the motion energy parameter of the t-th time point, wherein the reference standard deviation is negatively correlated with the motion energy parameter of the t-th time point;
determining a reference normal distribution curve as the reference mask based on the predicted heart rate and the reference standard deviation of the t-th time point, wherein a mean and a standard deviation of the reference normal distribution curve are respectively the predicted heart rate and the reference standard deviation of the t-th time point.

9. The method according to claim 1, wherein the step of determining the specific photoplethysmography spectrum based on the reference mask and the reference photoplethysmography spectrum comprises:
multiplying the reference mask by the photoplethysmography spectrum to generate the specific photoplethysmography spectrum.

10. A heart rate estimation device, comprising:
a storage circuit, storing a code;
a processor, coupled to the storage circuit and accessing the code to execute:
obtaining a reference photoplethysmography spectrum corresponding to a t-th time point, where t is a time index value;
obtaining a previous heart rate and a motion energy parameter corresponding to the t-th time point, and accordingly determining a predicted heart rate corresponding to the t-th time point;
determining a reference mask based on the predicted heart rate and the motion energy parameter of the t-th time point;
determining a specific photoplethysmography spectrum based on the reference mask and the reference photoplethysmography spectrum; and
estimating a heart rate corresponding to the t-th time point based on the specific photoplethysmography spectrum.

11. The device according to claim 10, wherein the reference photoplethysmography spectrum is a photoplethysmography spectrum after de-noising processing.

12. The device according to claim 10, wherein the processor executes:
obtaining an original motion energy parameter corresponding to the t-th time point;
obtaining a plurality of historical motion energy parameters corresponding to a (t-k)-th time point to a (t−1)-th time point, where k is a window length;
determining the motion energy parameter corresponding to the t-th time point based on the original motion energy parameter corresponding to the t-th time point and the historical motion energy parameters.

13. The device according to claim 12, wherein the motion energy parameter corresponding to the t-th time point is a weighted result of the original motion energy parameter and the historical motion energy parameters.

14. The device according to claim 10, wherein the previous heart rate is a heart rate corresponding to a (t−j)-th time point, where j is a positive integer.

15. The device according to claim 10, wherein the processor executes:
determining a reference value based on the previous heart rate and the motion energy parameter corresponding to the t-th time point; and
determining the predicted heart rate corresponding to the t-th time point based on the reference value, wherein the reference value is positively correlated with the predicted heart rate corresponding to the t-th time point.

16. The device according to claim 15, wherein the reference value is represented as:
A(t)=ACₜ*w1+LastHR*w2+c,
where ACₜ is the motion energy parameter corresponding to the t-th time point, LastHR is the previous heart rate, c is a constant, w1 and w2 are coefficients, 0≤w1≤1, and 0≤w2≤1.

17. The device according to claim 10, wherein the processor executes:
determining a reference standard deviation based on the motion energy parameter of the t-th time point, wherein the reference standard deviation is negatively correlated with the motion energy parameter of the t-th time point;
determining a reference normal distribution curve as the reference mask based on the predicted heart rate and the reference standard deviation of the t-th time point, wherein a mean and a standard deviation of the reference normal distribution curve are respectively the predicted heart rate and the reference standard deviation of the t-th time point.

18. The device according to claim 10, wherein the processor executes:
multiplying the reference mask by the photoplethysmography spectrum to generate the specific photoplethysmography spectrum.

19. A computer-readable storage medium, recording an executable computer program, wherein the executable computer program is loaded by a heart rate estimation device to execute:
obtaining a reference photoplethysmography spectrum corresponding to a t-th time point, where t is a time index value;
obtaining a previous heart rate and a motion energy parameter corresponding to the t-th time point, and accordingly determining a predicted heart rate corresponding to the t-th time point;
determining a reference mask based on the predicted heart rate and the motion energy parameter of the t-th time point;
determining a specific photoplethysmography spectrum based on the reference mask and the reference photoplethysmography spectrum; and
estimating a heart rate corresponding to the t-th time point based on the specific photoplethysmography spectrum.
